(19) United States
(12) Patent Application Publication
(10) Pub. No.: US 2025/0367087 A1
(43) Pub. Date: Dec. 4, 2025

(54) COMPOSITION FOR CLEANSING THE HAIR

(71) Applicant: L'OREAL, Paris (FR)

(72) Inventors: Yuanji GUO, Shanghai (CN); Jie DENG, Shanghai (CN); Xiuxia WANG, Shanghai (CN)

(73) Assignee: L'OREAL, Paris (FR)

(21) Appl. No.: 18/875,484

(22) PCT Filed: Jun. 21, 2022
(86) PCT No.: PCT/CN2022/100068

§ 371 Date: Dec. 16, 2024

(51) Int. Cl. A61K 8/368; A61K 8/46; A61K 8/49; A61Q 5/02

(52) U.S. Cl. CPC A61K 8/368; A61K 8/466; A61K 8/4926; A61Q 5/02; A61K 2800/34

(57) ABSTRACT

A composition for cleansing the hair comprises: a) at least one anionic surfactant; b) at least one alpha-hydroxy acid and at least one beta-hydroxy acid; c) at least one piroctone compound; and d) at least one alpha-olefin sulfonate. A process for cleansing the hair comprises applying the composition onto the hair, and then rinsing the hair with water after an optional period of exposure.

### TECHNICAL FIELD

The present invention relates to a composition for cleansing the hair. The present invention also relates to a process for cleansing the hair.

### BACKGROUND ART

Desquamating disorders of the scalp such as dandruff and seborrhoeic dermatitis are associated with the presence of a characteristic yeast known as Malassezia ovalis, which was previously known as Pityrosporum (P. ovale and P. orbiculare).

Moreover, it is known that this yeast is capable of changing its shape and metabolism. In particular, it can change into a filamentous form (Malassezia furfur) which is responsible for the inflammatory and pigmentary disorder known as Pityriasis versicolor.

The usual treatment for all these complaints involves using antifungal agents in a medium, such as a shampoo, a gel or a lotion, which is suitable for distributing these agents and depositing them on teguments.

The antifungal activity of these agents is limited; furthermore, the persistence of the antifungal activity is low. Thus, treatments using these antifungal agents are of very moderate or even low efficacy.

It is known that piroctone olamine is one of stronger anti-dandruff efficiency active, and lactic acid and salicylic acid has stronger peeling efficiency. However, piroctone olamine is very difficult to dissolve in a low pH system.

As commercial product, it is desired that the product is stable over time.

Thus, there is a need in the art to develop product for cleansing the scalp, which is effective in terms of antidandruff and stable over time.

### SUMMARY OF THE INVENTION

An object of the present invention is thus to develop compositions for cleansing the hair, which is effective in terms of antidandruff and stable over time.

Another object of the present invention is to provide a process for cleansing the scalp.

Thus, according to a first aspect, the present invention provides a composition for cleansing the hair comprising:
a) at least one anionic surfactant;
b) at least one alpha-hydroxy acid and at least one beta-hydroxy acid;
c) at least one piroctone compound; and
d) at least one alpha-olefin sulfonate.

Preferably, the composition according to the present invention is silicon-free.

According to a second aspect, the present invention provides a process for cleansing the hair comprising applying the composition as described above onto the hair, and then rinsing the hair with water after an optional period of exposure.

The inventors have found that the composition according to the present invention is effective in terms of dandruff and stable over time.

Other subjects and characteristics, aspects and advantages of the present invention will emerge even more clearly on reading the detailed description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well as optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

As used herein, the expression “at least one” used in the present description is equivalent to the expression “one or more”.

As used herein, “silicon free” means that no compound containing silicon is added on purpose and the content of the compound containing silicon in the composition is less than 0.5 wt. %, particularly less than 0.1 wt. %, relative to the total weight of the composition. In particular, there is no silicon in the composition.

According to the first aspect of the present invention, a composition for cleansing the hair comprises:
a) at least one anionic surfactant;
b) at least one alpha-hydroxy acid and at least one beta-hydroxy acid;
c) at least one piroctone compound; and
d) at least one alpha-olefin sulfonate.

### Anionic Surfactants

According to the first aspect of the present invention, the composition comprises at least one anionic surfactant.

The term “anionic surfactant” means a surfactant comprising, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be sulfate, and/or sulfonate surfactants.

It is understood in the present description that:
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The sulfonate anionic surfactants that may be used comprise at least one sulfonate function (—SO₃H or —SO₃⁻).

They may be selected from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds comprising from 6 to 30 carbon atoms, especially from 12 to 28, better still from 14 to 24 or even from 16 to 22 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfonate anionic surfactants are selected, alone or as a combination, from:
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates,
in particular in the form of alkali metal or alkaline-earth metal, ammonium or amino alcohol salts.

The sulfate anionic surfactants that may be used comprise at least one sulfate function (—OSO₃H or —OSO₃⁻).

They may be selected from the following compounds: alkyl sulfates, alkyl ether sulfates, alkylamido ether sulfates, alkylaryl polyether sulfates, monoglyceride sulfates; and also the salts of these compounds;
the alkyl groups of these compounds comprising from 6 to 30 carbon atoms, especially from 12 to 28, better still from 14 to 24 or even from 16 to 22 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfate anionic surfactants are selected, alone or as a combination, from:
C6-C24 alkyl sulfates and especially C12-C20 alkyl sulfates,
alkyl ether sulfates, especially of C6-C24 or even C12-C20, preferably comprising from 2 to 20 ethylene oxide units;
in particular in the form of alkali metal or alkaline-earth metal, ammonium or amino alcohol salts.

When the anionic surfactant is in salt form, the said salt may be selected from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are selected, alone or as a combination, from:
C6-C24 and especially C12-C20 alkyl sulfates;
C6-C24 and especially C12-C20 alkyl ether sulfates; preferably comprising from 2 to 20 ethylene oxide units;
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates;
in particular in the form of alkali metal such as sodium or alkaline-earth metal, ammonium or amino alcohol salts.

Preferably, the anionic surfactant is selected from sodium laureth sulfate, sodium lauryl sulfate, and a combination thereof.

Advantageously, the anionic surfactant is present in an amount ranging from 5 wt. % to 50 wt. %, preferably from 8 wt. % to 30 wt. %, more preferably from 10 wt. % to 20 wt. %, relative to the total weight of the composition.

### Hydroxy Acids

According to the first aspect of the present invention, the composition comprises at least one alpha-hydroxy acid and at least one beta-hydroxy acid.

### Alpha-Hydroxy Acids

The term “alpha-hydroxy acid” is understood to mean, according to the present invention, a carboxylic acid having at least one hydroxyl functional group occupying an alpha-position on said acid (carbon adjacent to a carboxylic acid functional group).

Suitable alpha-hydroxy acids includes glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid.

Preferably, the alpha-hydroxy acid is selected from lactic acid, glycolic acid, tartaric acid, mandelic acid, citric acid, and combinations thereof.

More preferably, the alpha-hydroxy acid in the composition consists of lactic acid.

Lactic acid, or 2-hydroxypropanoic acid, provides soft peeling on excess sebum, residues and loose dead cells around follicles and makes hair roots energized and lifted. In addition, lactic acid also boosts production of glycosaminoglycan (GAG) in the skin, improving the barrier function and moisturization of skin.

Advantageously, the alpha-hydroxy acid is present in an amount ranging from 0.5 wt. % to 20 wt. %, preferably from 1 wt. % to 15 wt. %, and more preferably from 2 wt. % to 10 wt. %, relative to the total weight of the composition.

### Beta-Hydroxy Acids

The term “beta-hydroxy acid” is understood to mean, according to the present invention, a carboxylic acid having a hydroxyl functional group and a carboxylic functional group separated by two carbon atoms.

Suitable beta-hydroxy acids include salicylic acid, propionic acid, beta-hydroybutyric acid, beta-hydroxy beta-methylbutyric acid, carnitine, derivatives thereof, and combinations thereof.

Preferably, the beta-hydroxy acid is selected from salicylic acid and its derivative of formula (I):

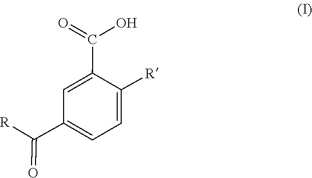

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;
R′ is a hydroxyl group or an ester functional group of the following formula:

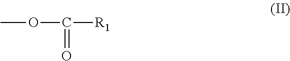

wherein R₁ is a linear or branched saturated or unsaturated aliphatic group having from 1 to 18 carbon atoms.

Preferred salicylic acid derivatives include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid.

More preferably, the beta-hydroxy acid is selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof.

Most preferably, the beta-hydroxy acid is salicylic acid.

Salicylic acid, or 2-hydroxybenzoic acid, is provided to the chemical peel composition to provide enhanced penetration of the composition into the skin. Salicylic acid penetrates deeper into the skin than alpha-hydroxy acids, such as lactic acid.

Advantageously, the beta-hydroxy acid is present in an amount ranging from 0.3 wt. % to 20 wt. %, preferably from 0.5 wt. % to 10 wt. %, and more preferably from 1 wt. % to 5 wt. %, relative to the total weight of the composition.

Advantageously, the ratio of alpha hydroxy acid to beta hydroxy acid is from 0.5-30, preferably from 1-15, and more preferably from 2-10. Preferably, the alpha hydroxy acid is lactic acid and the beta hydroxy acid is salicylic acid.

The inventors have found that the composition according to the present invention can remove the stubborn sebum effectively, in particular, when the composition contains lactic acid and salicylic acid or its derivative. Thus, the combination of surfactant and hydroxy acids deliver an even better long-lasting clean effect.

### Piroctone Compounds

The composition according to the present invention comprises at least one piroctone compound.

The piroctone compound for use in the present invention may include piroctone acid, primary, secondary and tertiary olamine salts of piroctone acid (such as the diethanolamine and triethanolamine salts), and mixtures thereof, preferably piroctone acid, primary olamine salt of piroctone acid (i.e. piroctone olamine, also known as Octopirox®) and mixtures thereof.

The piroctone compound useful in the present invention typically contains the structure defined by formula (III):

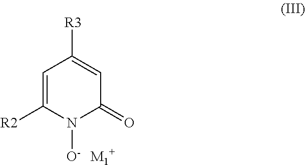

wherein R2 is selected from C1-C17 alkyl and C2-C17 alkenyl, R3 is selected from C1-4 alkyl, C2-4 alkenyl or alkynyl, hydrogen, phenyl or benzyl, and M1 is selected from hydrogen, monoethanolamine (MEA), diethanolamine (DEA), or triethanolamine (TEA).

Preferred R2 is (CH₃)₃CCH₂CH(CH₃)CH₂— and preferred R3 is a methyl.

More preferably, R2 is (CH₃)₃CCH₂CH(CH₃)CH₂—, R3 is a methyl and M1 is a hydrogen or MEA. Most preferably, R2 is (CH₃)₃CCH₂CH(CH₃)CH₂—, R3 is a methyl and M1 is hydrogen.

Particularly preferably, the composition according to the present invention comprises piroctone olamine.

Advantageously, the piroctone compound is present in an amount ranging from 0.1 wt. % to 10 wt. %, preferably from 0.3 wt. % to 5 wt. %, more preferably from 0.5 wt. % to 2 wt. %, relative to the total weight of the composition.

### Alpha-Olefin Sulfonates

The composition according to the present invention comprises at least one alpha-olefin sulfonate.

The alpha-olefin sulfonate (also called α-olefin sulfonate hereafter), preferably comprises 8 to 28 carbon atoms, especially from 10 to 24 carbon atoms, better still from 12 to 20 carbon atoms and in particular from 14 to 18 carbon atoms.

Said α-olefin sulfonates are known compounds and are described especially in Ullmann's Encyclopedia of Industrial Chemistry or in U.S. Pat. No. 8,211,850. These compounds are generally obtained by sulfonation of α-olefins.

Preferably, the alpha-olefin sulfonate is a linear alpha-olefin sulfonate (also called linear α-olefin sulfonate hereafter).

The linear alpha-olefin sulfonates according to the invention may be of formula (A):

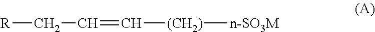

in which:
   R is a saturated linear alkyl radical comprising from 4 to 30 carbon atoms, especially from 6 to 20 carbon atoms, or even from 8 to 18 carbon atoms and better still from 10 to 14 carbon atoms;
   n is an integer between 0 and 10, preferably between 1 and 4 and better still n=1;
   M is a cosmetically acceptable cation, chosen especially from the ammonium cation, cations derived from alkali metals or alkaline-earth metals, cations derived from organic amines such as alkanolamines; preferably those derived from alkali metals; and especially Na⁺ or K⁺.

Preferably, R represents a linear alkyl radical comprising from 8 to 14 carbon atoms, especially from 10 to 12 carbon atoms.

Preferably, M is derived from an alkali metal, especially Na⁺ or K⁺.

Preferably, the linear olefin sulfonates according to the invention have the following formula:

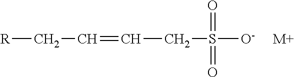

in which:
   R is a saturated linear alkyl radical comprising from 4 to 20 carbon atoms, especially from 6 to 18 carbon atoms, or even from 8 to 14 carbon atoms and better still from 10 to 12 carbon atoms;
   M is a cosmetically acceptable cation, chosen especially from the ammonium cation, cations derived from alkali metals or alkaline-earth metals, cations derived from organic amines such as alkanolamines; preferably those derived from alkali metals or alkali metals; and especially Na⁺ or K⁺.

Preferably, the linear α-olefin sulfonates according to the invention are chosen from linear α-olefin sulfonates comprising 8 to 28 carbon atoms, especially from 10 to 24 carbon atoms, better still from 12 to 20 carbon atoms, in particular from 14 to 18 carbon atoms; in particular of alkali metals, especially of sodium.

Commercial products that may especially be mentioned include those sold under the name Bio-Terge AS-40A or Bio-Terge AS-40HA by the company Stepan, or Calsoft AOS-40 by the company Pilot Chemical, or alternatively NANSA LSS 38/FL by the company INNOSPEC ACTIVE CHEMICALS.

Advantageously, the linear alpha-olefin sulfonate is present in an amount ranging from 0.1 wt. % to 20 wt. %, preferably from 0.3 wt. % to 15 wt. %, more preferably from 0.5 wt. % to 10 wt. %, most preferably from 0.5 wt. % to 3 wt. %, relative to the total weight of the composition.

### Aqueous Phase

The composition according to the present invention comprises an aqueous phase.

Preferably, the aqueous phase is a continuous phase.

The aqueous phase of the present invention comprises water.

The aqueous phase may also comprise water-miscible organic solvents (at room temperature of 20-25° C.), for instance polyols such as C2-C6 polyols, more particularly glycerin, hexylene glycol; glycol ethers (especially containing from 3 to 16 carbon atoms) such as mono-, di- or tripropylene glycol (C1-C4)alkyl ethers, mono-, di- or triethylene glycol (C1-C4)alkyl ethers, and combinations thereof.

Advantageously, the aqueous phase is present in an amount ranging from 60 wt. % to 90 wt. %, preferably from 65 wt. % to 85 wt. %, relative to the total weight of the composition.

### Additional Ingredients

The composition according to the present invention may also comprise any other additional ingredient that is usually used in the field of self-cleaning products, in particular shampoos.

The skilled in the art can select the amount of the additional ingredients so as not to adversely impact the final use of the composition according to the present invention.

For example, such additional ingredients include pH adjusting agents, preserving agents, antioxidants, fragrances, electrolytes and stabilizers, plant extracts, proteins, amino acids, vitamins, glycols, emollients, and combinations thereof.

Non-limiting examples of pH adjusting agents includes potassium acetate, potassium hydroxide, sodium carbonate, sodium hydroxide, phosphoric acid, succinic acid, sodium citrate, sodium hydrogen carbonate, and combinations thereof.

The pH adjusting agent is used in an amount so that the pH of the composition of the present invention is less than or equal to 6.5, preferably from 2 to 6.5, more preferably from 3.5 to 4.5.

According to a preferred embodiment, the present invention provides a composition for cleansing the hair comprising, relative to the total weight of the composition:
a) from 10 wt. % to 20 wt. % of at least one anionic surfactant;
b) from 2 wt. % to 10 wt. % of lactic acid and from 1 wt. % to 5 wt. % of at least one beta-hydroxy acid selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof;
c) from 0.5 wt. % to 2 wt. % of piroctone olamine; and
d) from 0.5 wt. % to 3 wt. % of at least one alpha-olefin sulfonate of formula:

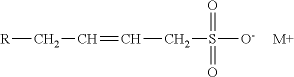

in which:
R is a saturated linear alkyl radical comprising from 4 to 20 carbon atoms, especially from 6 to 18 carbon atoms, or even from 8 to 14 carbon atoms and better still from 10 to 12 carbon atoms;
   M+ is selected from Na⁺ or K⁺,
wherein the pH of the composition is from 3.5 to 4.5.

### Preparation and Use

The composition according to the present invention can be prepared by mixing ingredients a) to d), as essential ingredients, as well as additional ingredient(s), as explained above.

The method and means to mix the above essential and optional ingredients are not limited. Any conventional method and means can be used to mix the above essential and optional ingredients to prepare the composition according to the present invention.

The composition according to the present invention can have a gel-like texture.

According to the second aspect of the present invention, a process for cleansing the hair comprising applying the composition as described above onto the hair, and then rinsing with water after an optional period of exposure.

The following examples are given by way of illustration of the present invention and shall not be interpreted as limiting the scope.

### EXAMPLES

Main raw materials used, trade names and supplier thereof are listed in Table 1.

TABLE 1

| INCI Name | Trade Name | Supplier |
|---|---|---|
| SODIUM LAURETH | TEXAPON N 701 | BASF |
| SULFATE | | |
| SODIUM C14-16 | NANSA LSS 38/FL | INNOSPEC |
| OLEFIN | | ACTIVE |
| SULFONATE | | CHEMICALS |
| LACTIC ACID | L(+)-LACTIC ACID | JUNGBUNZLAUER |
| | 90% HEAT STABLE | |
| | PERSONAL CARE | |
| | GRADE | |
| SALICYLIC ACID | SALICYLIC ACID USP | ALTA |
| | | LABORATORIES |
| PIROCTONE | PIROCTONE | YANTAI AURORA |
| OLAMINE | OLAMINE | CHEMICAL |
| Sodium Hydroxide | SODIUM | PT. MULIA AGUNG |
| | HYDROXIDE 48% | CHEMINDO |

### Invention Examples 1-2 and Comparative Example 1

Shampoos according to invention examples (IE.) 1-2 and comparative example (CE.) 1 were prepared with the ingredients listed in Table 2 (the contents are expressed as weight percentages of active material with regard to the total weight of each shampoo, unless otherwise indicated):

TABLE 2

| Components | IE. 1 | IE. 2 | CE. 1 |
|---|---|---|---|
| SODIUM LAURETH SULFATE | 15 | 15 | 15 |
| SODIUM C14-16 OLEFIN SULFONATE | 1 | 1 | / |
| LACTIC ACID | 5.4 | 5.4 | 5.4 |
| SALICYLIC ACID | 1.5 | 1.5 | 1.5 |
| PIROCTONE OLAMINE | 0.8 | 0.5 | 0.8 |
| SODIUM HYDROXIDE | 1.27 | 1.27 | 1.27 |
| WATER | QS100 | QS100 | QS100 |
| pH | 3.8 | 3.8 | 3.8 |

Shampoos of invention examples (IE.) 1-2 are compositions according to the present invention.

Shampoo of comparative example (CE.) 1 does not comprise alpha-olefin sulfonate.

### Preparation Procedure:

Each shampoo was prepared as follows, taking shampoo of invention example 1 for illustration.
1). adding SODIUM LAURETH SULFATE and SODIUM C14-16 OLEFIN SULFONATE in water and stirring at room temperature to obtain a homogeneous mixture,
2). adding LACTIC ACID and SALICYLIC ACID and PIROCTONE OLAMINE into the homogeneous mixture and stirring at room temperature,
3). adding sodium hydroxide to adjust the pH value to 3.8, to obtain the shampoo.

### Evaluation:

Stability and antidandruff efficiency of each composition prepared above were characterized.

### Stability

The appearance of each composition prepared above upon storage at 4° C., room temperature, 37° C., or 45° C. for 2 months was observed to evaluate the stability of the composition.

If the appearance of a composition is transparent after two months of storage at each of 4° C., room temperature, 37° C., and 45° C., then the stability is evaluated to be ‘Good’. If there is precipitate (crystal) in the composition after two months of storage at any of 4° C., room temperature, 37° C., and 45° C., then the stability is evaluated to be ‘Not Good’.

The results on viscosity and stability were summarized in Table 3.

TABLE 3

| Properties | IE. 1 | IE. 2 | CE. 1 |
|---|---|---|---|
| Appearance | Transparent | Transparent | Precipitate |
| Stability | Good | Good | Not Good |

### Antidandruff Efficiency

The antidandruff efficiency of compositions of invention examples 1 and 2 as well as HS shampoo from Procter & Gamble Company was evaluated according to QB/T 2738-2012: test methods for evaluating daily chemical products in antibacterial and bacteriostatic efficacy-7.3 the test method of bacteriostatic activity for bacteriostatic daily chemical products (Quantitative suspension test). HS shampoo contains 0.8 wt. % of OCT (PIROCTONE OLAMINE) and is well known for its antidandruff efficiency in the market. In addition, HS shampoo contains small amount of citric acid to adjust the pH value to around 5.7 to control the preservative system, and HS shampoo does not contain beta-hydroxy acid.

Test organism: Malassezia fufur ATCC 44344.

The antidandruff efficiency was summarized in Table 4.

TABLE 4

| | | IE. 1 | IE. 2 | HS shampoo |
|---|---|---|---|---|
| Antidandruff efficiency (%) | 5 min | 99.9 | 99.9 | 26 |

The results show that compositions of invention examples 1 and 2 have better antidandruff efficiency as compared with HS shampoo from Procter & Gamble.

1. A composition for cleansing the hair comprising:
a) at least one anionic surfactant;
b) at least one alpha-hydroxy acid and at least one beta-hydroxy acid;
c) at least one piroctone compound; and
d) at least one alpha-olefin sulfonate.

2. The composition according to claim 1, wherein the anionic surfactant is selected from
C6-C24 alkyl sulfates;
C6-C24 alkyl ether sulfates;
C6-C24 alkylsulfosuccinates;
C6-C24 alkyl ether sulfosuccinates;
C6-C24 acylisethionates;
in the form of alkali metal or alkaline-earth metal, ammonium or amino alcohol salts.

3. The composition according to claim 1, wherein the anionic surfactant is present in an amount ranging from 5 wt. % to 50 wt. % relative to the total weight of the composition.

4. The composition according to claim 1, wherein the alpha-hydroxy acid is selected from the group consisting of lactic acid, glycolic acid, tartaric acid, mandelic acid, citric acid, and combinations thereof.

5. The composition according to claim 1, wherein the alpha-hydroxy acid is present in an amount ranging from 0.5 wt. % to 20 wt. % relative to the total weight of the composition.

6. The composition according to claim 1, wherein the beta-hydroxy acid is selected from salicylic acid and its derivative of the formula: 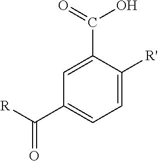
wherein R is a linear, branched or cyclic saturated aliphatic group or an aliphatic unsaturated group containing one or a number of double bonds, optionally conjugated, these groups containing from 2 to 22 carbon atoms optionally substituted by at least one substituent selected from the group consisting of (a) halogen atoms, (b) the trifluoromethyl group, (c) hydroxyl groups in the free form or esterified by an acid having from 1 to 6 carbon atoms and (d) a carboxyl functional group which is free or esterified by a lower alcohol having from 1 to 6 carbon atoms;
R′ is a hydroxyl group or an ester functional group of the following formula: 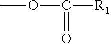
wherein R₁ is a linear or branched saturated or unsaturated aliphatic group having from 1 to 18 carbon atoms.

7. The composition according to claim 1, wherein the beta-hydroxy acid is present in an amount ranging from 0.3 wt. % to 20 wt. % relative to the total weight of the composition.

8. The composition according to claim 1, wherein the piroctone compound is of formula (III): 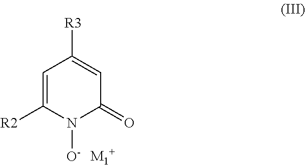
wherein R2 is selected from C1-C17 alkyl and C2-C17 alkenyl, R3 is selected from C1-4 alkyl, C2-4 alkenyl or alkynyl, hydrogen, phenyl or benzyl, and M1 is selected from hydrogen, monoethanolamine (MEA), diethanolamine (DEA), or triethanolamine (TEA).

9. The composition according to claim 1, wherein the piroctone compound is present in an amount ranging from 0.1 wt. % to 10 wt. % relative to the total weight of the composition.

10. The composition according to claim 1, wherein the alpha-olefin sulfonate is selected from linear alpha-olefin sulfonates of formula (A): 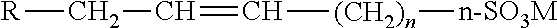
wherein
R is a saturated linear alkyl radical comprising from 4 to 30 carbon atoms;
n is an integer between 0 and 10;
M is a cosmetically acceptable cation selected from the group consisting of an ammonium cation, cations of alkali metals, cations of alkaline-earth metals, and cations of organic amines.

11. The composition according to claim 10, wherein the alpha-olefin sulfonate is selected from those of the following formula: 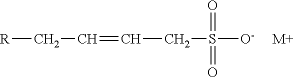
wherein
R is a saturated linear alkyl radical comprising from 4 to 20 carbon atoms;
M is a cosmetically acceptable cation selected from the group consisting of an ammonium cation, cations of alkali metals, cations of alkaline-earth metals, and cations of organic amines.

12. The composition according to claim 1, wherein the alpha-olefin sulfonate is present in an amount ranging from 0.1 wt. % to 20 wt. % relative to the total weight of the composition.

13. The composition according to claim 1, wherein the pH of the composition is from 2 to 6.5.

14. The composition according to claim 1, comprising, relative to the total weight of the composition:
a) from 10 wt. % to 20 wt. % of at least one anionic surfactant;
b) from 2 wt. % to 10 wt. % of lactic acid and from 1 wt. % to 5 wt. % of at least one beta-hydroxy acid selected from the group consisting of salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof;
c) from 0.5 wt. % to 2 wt. % of piroctone olamine; and
d) from 0.5 wt. % to 3 wt. % of at least one alpha-olefin sulfonate of formula: 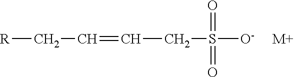
wherein
R is a saturated linear alkyl radical comprising from 4 to 20 carbon atoms;
M+ is Na⁺ or K⁺, and
wherein the pH of the composition is from 3.5 to 4.5.

15. The composition of claim 1, which is silicon-free.

16. A process for cleansing the hair comprising applying the composition according to claim 1 onto the hair, and then rinsing the hair with water after an optional period of exposure.
